(19) United States
(12) Patent Application Publication
(10) Pub. No.: US 2026/0298808 A1
(43) Pub. Date: Oct. 1, 2026

(54) METHOD FOR DEVELOPING TISSUE-SPECIFIC COLOR CHART

(71) Applicant: Purdue Research Foundation, West Lafayette, IN (US)

(72) Inventors: Young KIM, West Lafayette, IN (US); Sang Mok PARK, West Lafayette, IN (US); Yuhyun JI, West Lafayette, IN (US); Semin KWEON, West Lafayette, IN (US); Jungwoo LEEM, West Lafayette, IN (US)

(73) Assignee: Purdue Research Foundation, West Lafayette, IN (US)

(21) Appl. No.: 19/489,828

(22) PCT Filed: Apr. 20, 2024
(86) PCT No.: PCT/US2024/025608

§ 371 Date: Dec. 3, 2025

Related U.S. Application Data
(60) Provisional application No. 63/524,318, filed on Jun. 30, 2023.
(60) Provisional application No. 63/524,352, filed on Jun. 30, 2023.

(51) Int. Cl. G01N 21/27; G01N 33/483; G06T 7/00

(52) U.S. Cl. CPC G01N 21/274; G01N 33/483; G06T 7/0014; G06T 2207/20081

(57) ABSTRACT

A method of generating a ground truth color chart including color patches for biological imaging and colorimetric bioassays is disclosed which includes using a mathematical relationship associated with a tissue of interest expressing a relationship between intensity as a function of wavelength, the received mathematical relationship having a plurality of parameters, synthesizing a plurality of synthesized spectra based on the mathematical relationship, converting each of the plurality of synthesized spectra to a predetermined color space under a predetermined illuminant, thereby generating a digital tissue-specific gamut, identifying a plurality of digital gamut points within the digital tissue-specific gamut based on a predetermined criterion, and printing a tissue-specific gamut based on the digital tissue-specific gamut thus establishing a ground truth color chart having a plurality of color patches, whereby each of the plurality of digital gamut points corresponds to an associated printed color patch using a predetermined printing calibration.

### CROSS-REFERENCE TO RELATED APPLICATIONS

The present non-provisional patent application is related to and claims the priority benefit of U.S. Provisional patent application Ser. 63/524,318, filed Jun. 30, 2023, and of U.S. Provisional patent application Ser. 63/524,352, filed Jun. 30, 2023, the contents of each of which are hereby incorporated by reference in their entirety into the present disclosure.

### STATEMENT REGARDING GOVERNMENT FUNDING

This invention was made with government support under R01EB033788 awarded by the National Institutes of Health. The government has certain rights in the invention.

### TECHNICAL FIELD

The present disclosure generally relates to developing color standard or reference charts and specifically for developing color charts for diagnostic applications.

### BACKGROUND

This section introduces aspects that may help facilitate a better understanding of the disclosure. Accordingly, these statements are to be read in this light and are not to be understood as admissions about what is or is not prior art.

A photograph can provide diagnostic information beyond a mere visual representation. The primary challenge is to achieve color accuracy, which is referred to as the ability to define standardized colors of a sample and accurately detect them across diverse photo acquisition settings. Photographs acquired using a digital camera exhibit detrimental variations in colors, depending on device models, light conditions, and file formats. Color accuracy in medical imaging, color consistency in machine vision, and color constancy in human perception are distinct yet interrelated aspects of color science and management. The importance of color consistency is well recognized in machine vision and general photography, which focuses on color correction and color reproduction. Color constancy in human perception primarily focuses on achieving identical perceptual response. However, there is a lack of studies to realize color accuracy in medical imaging. Recently, the onboard cameras of mobile devices have been extensively used for medical photography, including telemedicine and mobile health (mHealth) applications. Healthcare professionals now consider the acquisition of photos using mobile devices (i.e., smartphones and tablets) to be a mandatory part of healthcare practice because diagnostic photography plays a key role in a variety of digital health applications. As a result, there is an urgent need for implementing color accuracy in diagnostic photography.

Reproducing original colors is challenging in photography, printing, and electronic displays because of intrinsic color distortions during image acquisition, processing, and output. The existing color calibration or correction methods using the conventional color charts are inadequate to achieve high fidelity of color accuracy for diagnostic purposes. First, generating a universal color gamut that can exactly reproduce all colors is challenging. Optimizing a unique color gamut is necessary, but identifying relevant yet subtle color derivatives is not straightforward without spectroscopic analyses, which enable to augment homogeneous data for ideal gamut determination. Second, color undersampling in the conventional color charts fundamentally reduces the degree of color accuracy. Color correction and calibration computations with a limited number of reference colors are prone to random and systematic errors (e.g., Gaussian, impulse, photon, or speckle noise) in a variety of photo acquisitions. Incorporating more reference colors allows us to use advanced machine learning techniques that can outperform the conventional computations. Third, regression-based color correction computations (also known as lookup table and interpolation) commonly used in machine vision require manual fine-tuning, depending on photo acquisition settings.

In digital photography, color appearance is considerably affected by a variety of factors, settings, and conditions. Reference is made to FIG. 1A wherein representative digital photos of biological tissue (e.g., palpebral conjunctiva) captured under five various white-light illumination conditions are provided including LEDs with color temperature of 3000 K, 4300 K, and 5800 K as well as fluorescent tube light. Color accuracy in diagnostic photography refers to the ability to detect the absolute colors across various photo acquisition settings. From a device standpoint, a digital trichromatic camera (three-color image sensor) has unique red-green-blue (RGB) spectral response functions (also known as the spectral sensitivity) as a function of the wavelength of light A. Referring to FIG. 1B, model-specific RGB spectral response functions lead to device-dependent color mapping of several popular low- and high-end smartphones with ANDROID and iOS operating systems including APPLE IPHONE 12 PRO, APPLE IPHONE SE, SAMSUNG GALAXY S21, and SAMSUNG GALAXY A52. Notably, the RGB spectral responses of smartphone cameras exhibit significant model-specific variations as shown in FIGS. 1C and 1D, resulting in notoriously device-dependent RGB color values. From an illumination standpoint, different types of white light sources have distinct spectral profiles in otherwise grossly white-appearing light conditions as shown in FIGS. 1E and 1F. Diverse spectral characteristics of light sources fundamentally limit color management (e.g., white balancing), resulting in light condition-specific RGB color values as shown in FIG. 1G. From file format and bit depth (color depth) standpoints, the degree of color compression is significantly influenced by the image file format as shown in FIGS. 1H and 1I. JPEG, the most commonly used file format (8-bit depth), uses lossy compression to reduce file size. RAW (also known as DNG) minimizes data compression and rendering. Recent high-end smartphones provide Pro Mode or ProRAW for 10-bit depth in each RGB channel. In telemedicine settings (FIG. 1H), live, two-way video conferencing between a healthcare provider and patient introduces additional diversity in file formats (e.g., MP4). Overall, diverse photo acquisition scenarios pose a fundamental challenge for color accuracy in diagnostic photography.

Therefore, there is an unmet need for a novel method that can be used to generate a tissue-specific ground truth color chart that can be used with biological tissue-specific photos.

### SUMMARY

A method of generating a ground truth color chart including color patches for biological imaging and colorimetric bioassays is disclosed. The method includes using a mathematical relationship associated with a tissue of interest expressing a relationship between intensity as a function of wavelength, the received mathematical relationship having a plurality of parameters each with an established range, synthesizing a plurality of synthesized spectra based on the mathematical relationship, converting each of the plurality of synthesized spectra to a predetermined color space under a predetermined illuminant, thereby generating a digital tissue-specific gamut, identifying a plurality of digital gamut points within the digital tissue-specific gamut based on a predetermined criterion, and printing a tissue-specific gamut based on the digital tissue-specific gamut thus establishing a ground truth color chart having a plurality of color patches, whereby each of the plurality of digital gamut points corresponds to an associated printed color patch using a predetermined printing calibration.

In the above method, the predetermined illuminant is International Commission on Illumination (CIE) illuminant E.

In the above method, the plurality of types of tissue includes palpebral conjunctiva.

In the above method, the tissue of interest includes skin.

In the above method, the tissue of interest includes teeth.

In the above method, the tissue of interest includes tongue.

In the above method, the tissue of interest includes eye.

In the above method, the tissue of interest includes bulbar conjunctiva.

In the above method, tissue of interest includes whole blood.

In the above method, tissue of interest includes eye tears.

In the above method, tissue of interest includes nailbed.

In the above method, tissue of interest includes external eye.

In the above method, tissue of interest includes stool.

In the above method, the step of synthesizing a plurality of synthesized spectra is based on a plurality of parameter data points associated with each of the parameters of the plurality of parameters.

In the above method, one or more of the plurality of parameters are associated with whole blood and one or more of the parameters are associated with peripheral tissue.

In the above method, the mathematical relationship is expressed as:

$$I ( λ ) = [ b + ( 1 - b ) ( {{a}_{11} [ \frac{λ}{{λ}_{0}} ]}^{{a}_{12}} + {{a}_{13} [ \frac{λ}{{λ}_{0}} ]}^{{a}_{14}} ) ] {e}^{\{ - {a}_{2} {μ}_{a}^{Total} ( λ ) \times [ b + ( 1 - b ) \frac{{a}_{3} ( 1 - exp [ - {a}_{4} {μ}_{a}^{Total} ( λ ) ] )}{{a}_{4} {μ}_{a}^{Total} ( λ )} ] \}} ,$$

wherein
I represents spectral intensity,
λ represents wavelength,
the plurality of parameters are a₁₁, a₁₂, a₁₃, a₁₄, a₂, a₃, a₄, a₅, and a₆, wherein:
a₁₁, a₁₂, a₁₃, and a₁₄ represent tissue scattering contributions,
a₂ represents optical pathlength,
a₃ represents blood volume fraction,
a₄ represents effective blood vessel diameter in peripheral tissue,
a₅ represents blood hemoglobin (Hgb) content,
a₆ represents blood oxygen saturation,
λ₀ represents a constant for wavelength normalization,

$${μ}_{a}^{Total} ( λ )$$

represents total absorption coefficient including oxygenated and deoxygenated Hgb, wherein:

$${μ}_{a}^{Total} ( λ ) = {a}_{5} [ {a}_{6} {ε}_{{HgbO}_{2}} ( λ ) + ( 1 - {a}_{6} ) {ε}_{Hgb} ( λ ) ] ,$$

wherein b represents a flag wherein when b=0, the mathematical relationship represents peripheral tissue and when b=1, the mathematical relationship represents whole blood,
ε_{HgbO}_{₂}(λ) represents extinction coefficient of oxygenated Hgb, and
ε_{Hgb}(λ) represents extinction coefficient of deoxygenated Hgb.

In the above method, the tissue of interest includes whole blood and palpebral conjunctiva; a₁₁ is represented by a range of about 0 to about 1 for b=0; and not applicable for b=1; a₁₂ is represented by a range of about −1 to about 2 for b=0; and not applicable for b=1; a₁₃ is represented by a range of about 0 to about 1 for b=0; and not applicable for b=1; a₁₄ is represented by a range of about −1 to about 2 for b=0; and not applicable for b=1; a₂×a₃ is represented by a range of about 0.02 to about 0.1 for b=0; a₂ is about 0.1, and a₃ is not applicable for b=1; a₄ is represented by a range of about 5 to about 70 for b=0; and not applicable for b=1; a₅ is represented by a range of about 4 to about 20; and a₆ is represented by a range of about 0 to about 1.

In the above method, a₁₁, a₁₂, a₁₃, a₁₄, a₂, a₃, a₄, a₅, and a₆ are related to peripheral tissue for when b=0.

In the above method, a₂, a₅, and a₆ are related to whole blood for when b=1, where

$${μ}_{a}^{Tota1} ( λ )$$

represents total absorption coefficient including other light absorbing pigments and molecules in biological samples.

In the above method, the predetermined color space is RGB.

In the above method, the predetermined color space is CIE XYZ.

In the above method, the predetermined color space is CIE RGB.

In the above method, the predetermined color space is CIE YUV.

In the above method, the predetermined color space is CIE UVW.

In the above method, the predetermined color space is CIE LAB.

In the above method, the predetermined color space is CIE LUV.

In the above method, the predetermined color space is HSL.

In the above method, the predetermined color space is HSV.

In the above method, the predetermined color space is HCL.

In the above method, the predetermined color space is LMS.

Another method of generating a ground truth color chart for colorimetric bioassays is also disclosed. The method includes generating a digital bioassay-specific gamut using a predetermined color space under a predetermined illuminant for a bioassay-specific test strip having a plurality of reference color panels, identifying a plurality of digital gamut points within the digital bioassay-specific gamut based on a predetermined criterion, and printing a bioassay-specific gamut based on the digital bioassay-specific gamut thus establishing a ground truth color chart having a plurality of color patches, whereby each of the plurality of digital gamut points corresponds to an associated printed color patch using a predetermined printing calibration.

In the above method, the predetermined illuminant is International Commission on Illumination (CIE) illuminant E.

In the above method, the bioassay-specific test strip is for urine.

In the above method, the bioassay-specific test strip is for blood.

In the above method, the bioassay-specific test strip is for eye tears.

In the above method, the bioassay-specific test strip is for saliva.

In the above method, the bioassay-specific test strip is for stool.

In the above method, the bioassay-specific test strip is for body fluid.

In the above method, the bioassay-specific test strip is for biological solutions.

In the above method, the predetermined color space is RGB.

In the above method, the predetermined color space is CIE XYZ.

In the above method, the predetermined color space is CIE RGB.

In the above method, the predetermined color space is CIE YUV.

In the above method, the predetermined color space is CIE UVW.

In the above method, the predetermined color space is CIE LAB.

In the above method, the predetermined color space is CIE LUV.

In the above method, the predetermined color space is HSL.

In the above method, the predetermined color space is HSV.

In the above method, the predetermined color space is HCL.

In the above method, the predetermined color space is LMS.

### BRIEF DESCRIPTION OF FIGURES

FIG. 1A provides representative digital photos of biological tissue (e.g., palpebral conjunctiva) captured under five various white-light illumination conditions are provided including LEDs with color temperature of 3000 K, 4300 K, and 5800 K as well as fluorescent tube light.

FIG. 1B provides model-specific RGB spectral response functions which lead to device-dependent color mapping of several popular low- and high-end smartphones with ANDROID and iOS operating systems including APPLE IPHONE 12 PRO, APPLE IPHONE SE, SAMSUNG GALAXY S21, and SAMSUNG GALAXY A52.

FIGS. 1C and 1D provide device-dependent RGB color values.

FIGS. 1E and 1F provides different types of white light sources which have distinct spectral profiles in otherwise grossly white-appearing light conditions.

FIG. 1G provides diverse spectral characteristics of light sources which fundamentally limit color management (e.g., white balancing), resulting in light condition-specific RGB color values.

FIGS. 1H and 1I provide degree of color compression (FIG. 1H: schematic of file compressions) significantly influenced by the image file format (FIG. 1I: DNG, JPEG, and MP4).

FIG. 2 is a flowchart that outlines the two approaches and their common convergence toward generating a tissue-specific ground truth color chart.

FIG. 3A is a hyperspectral image capture device captures a line-scan from an area of interest (e.g., the inner eyelid).

FIG. 3B is the resulting hyperspectral image in which this image is subdivided into three parts along a vertical (y-axis) while the horizontal axis (x-axis) is the associated wavelength in nm.

FIG. 3C provides the subdivisions in FIG. 3B in the form of spectra.

FIG. 3D provides spectra shown in FIG. 3C into an average spectrum.

FIGS. 4A and 4B provide 12,240 spectral datapoints generated from peripheral tissue (FIG. 4A) and 10,000 spectral datapoints generated of whole blood (FIG. 4B) in the visible range by varying parameters discussed below.

FIG. 4C provides converted synthesized spectra to a CIE XYZ tristimulus value resulting in generation of a digital tissue-specific gamut.

FIG. 4D provides division between the 12,240 and the 10,000 spectral datapoints of FIGS. 4A and 4B based on the plurality of parameters discussed below.

FIGS. 5A and 5B provide number of points, e.g., 116, inside and outside of a digital Hgb gamut—these points represent 116 reference color patches including 100 CIE xy chromaticity values that are uniformly distributed inside the Hgb gamut as well as 12 primary colors and four black and white colors to interpolate colorimetric outliers outside the Hgb gamut and to compute the spectral response functions of the image capture device, e.g., a smartphone camera.

FIGS. 5C and 5D provide printed tissue-specific gamut determined based on the digital tissue-specific gamut shown in FIGS. 5A and 5B, whereby each of the plurality of digital gamut points corresponds to an associated printed gamut point thereby establishing an error signal corresponding to a plurality of CMYK (cyan, magenta, yellow, and key (black)) values used for printing the associated printed gamut points.

FIG. 5E is a color chart for each of the printed gamut points in FIGS. 5C and 5D.

FIG. 6 provides a non-calibrated image of a bioassay-specific test strip along with a conventional color chart (e.g., Macbeth ColorChecker or X-Rite ColorChecker), wherein the color chart has m color patches, e.g., m=24.

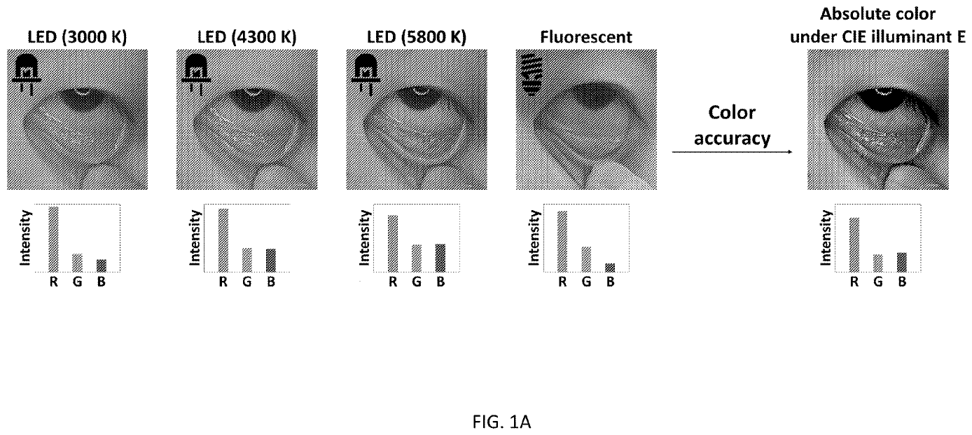

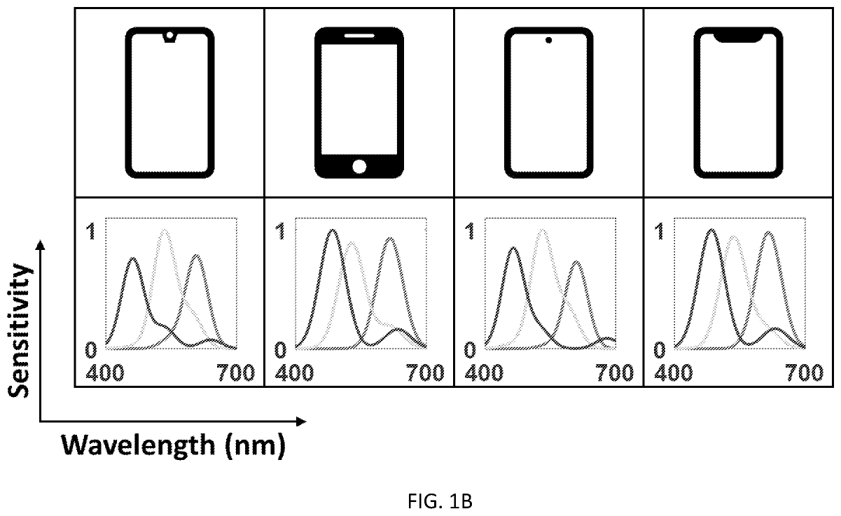

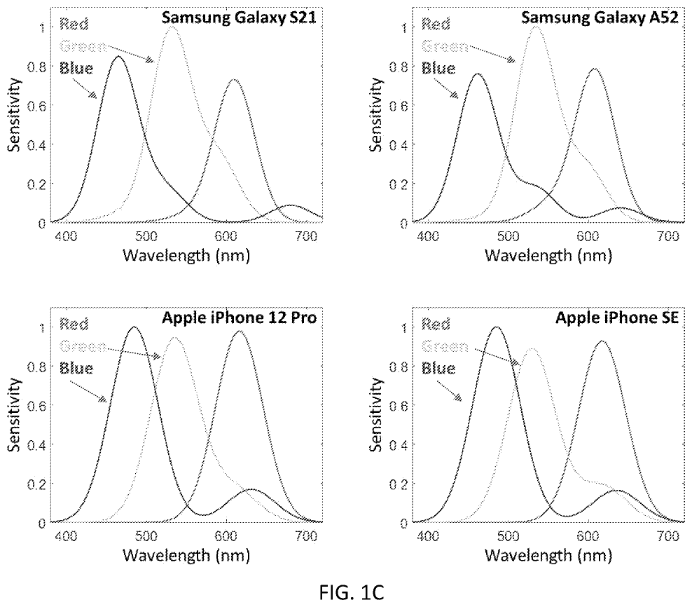

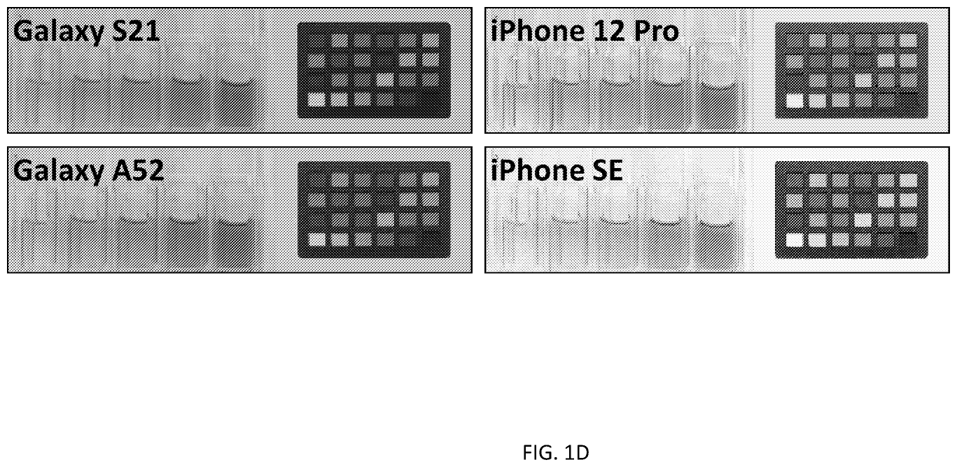

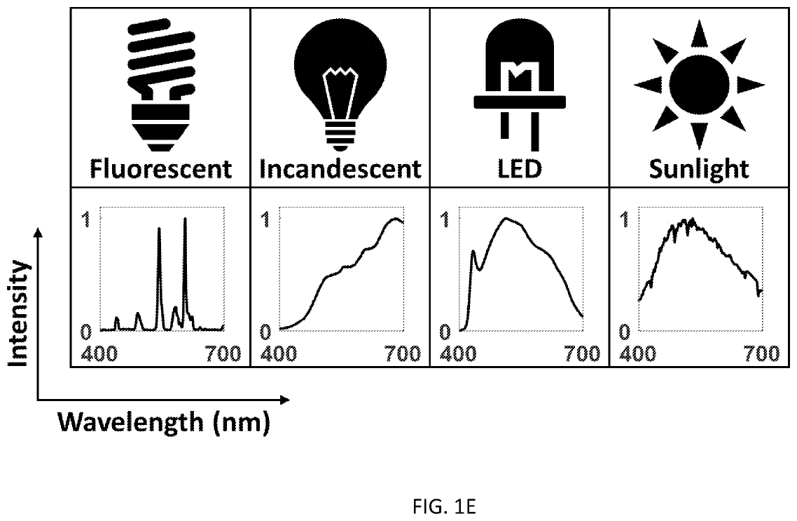

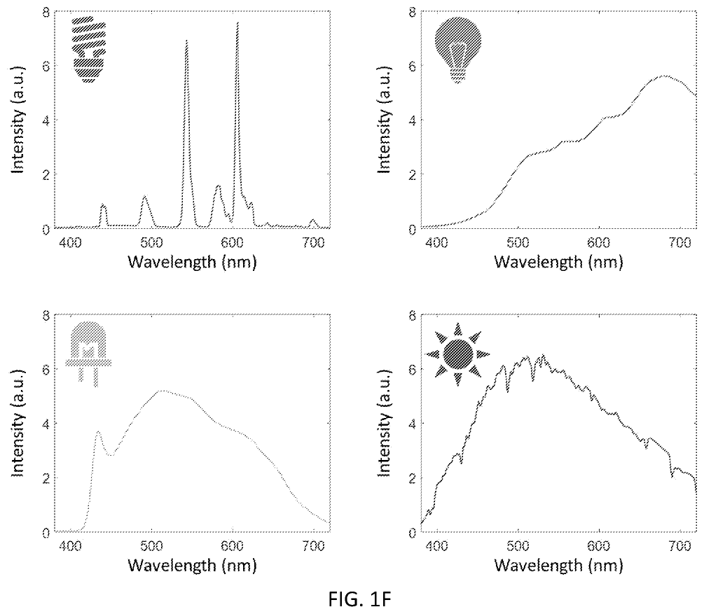

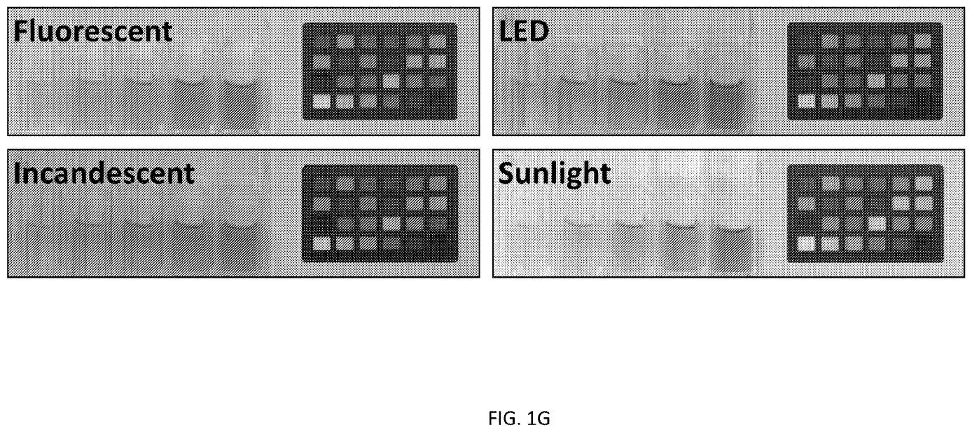

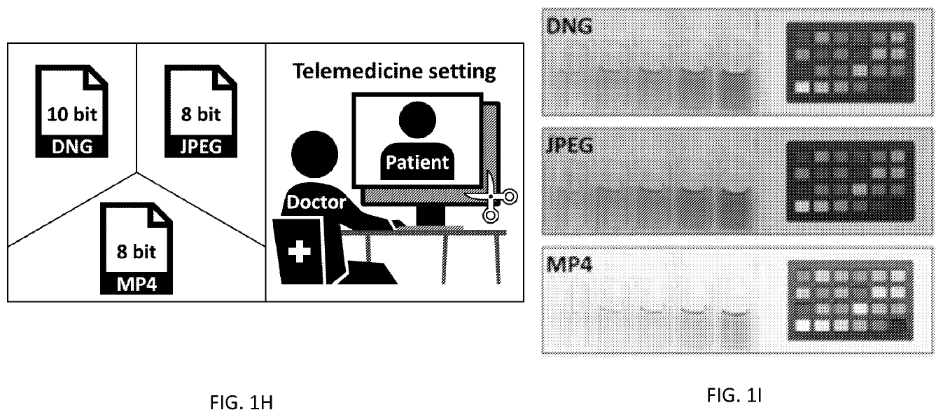

### DETAILED DESCRIPTION

For the purposes of promoting an understanding of the principles in the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

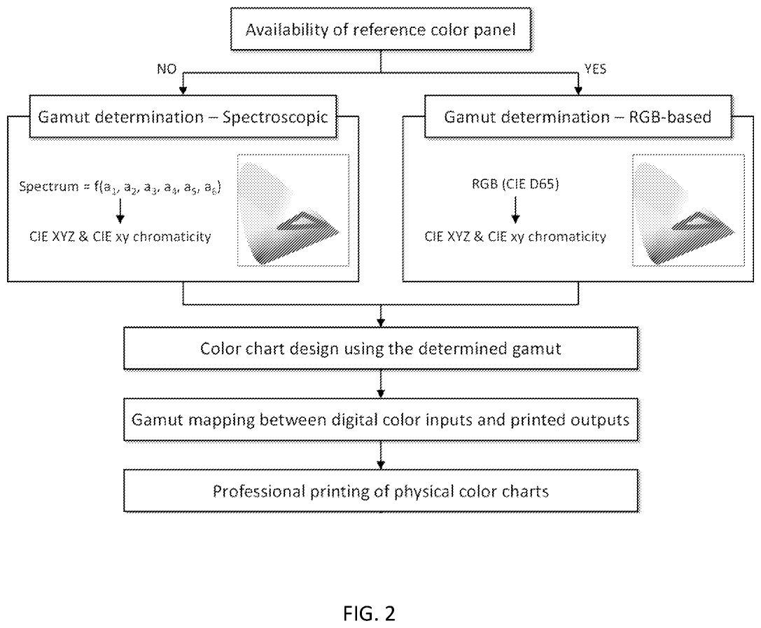

A novel method is disclosed herein that can be used to generate a tissue-specific ground truth color chart that can be used with biological tissue-specific images. Towards this end two approaches are discussed herein. These two approaches converge to generate a ground truth color chart that is specific to a tissue. Referring to FIG. 2, a flowchart is provided that outlines the two approaches and their common convergence toward generating a tissue-specific ground truth color chart. Two different types of color gamut determination are shown in FIG. 2. The first one is gamut determination based on spectral data, while the second is RGB (red-green-blue)-based.

Identification of a color gamut is required to achieve accurate and precise ground truth color charts in diagnostic colorimetric applications in bioimaging and bioassays. Commonly used conventional color gamuts (e.g., sRGB) are too broad to offer accurate and precise color corrections. Depending on the availability of commercially available reference color panels for a biological sample or bioassay strip of interest, two different approaches are disclosed herein to determine the specific gamut.

First, spectroscopic gamut determination (e.g., blood hemoglobin and peripheral tissue (e.g., inner eyelid)) is discussed. We can use parametric biological tissue spectral modeling if reference color panels for a biological sample of interest are unavailable. By generating spectral profiles using biological tissue spectral modeling, we determine a specific color gamut. For example, a color gamut specific to blood hemoglobin (Hgb) and peripheral tissue perfusion can be determined. Physiologically relevant ranges of model parameters are varied to generate possible spectra, from which CIE xy chromaticity values are calculated. This allows for the determination of a specific unique and narrow triangular color gamut in the CIE xy chromaticity diagram. Instead of parametric biological tissue spectral modeling, we can also use Monte Carlo simulations and approximation of radiative transport (e.g., diffusion and Born) combined with specific optical properties or relevant biological ranges. This is based on the idea that spectral profiles can be used to classify the types of biological samples.

The second type of gamut determination is based on RGB data. This type is generally seen in strip test (e.g., colorimetric strip tests (e.g., urine test strips)). We can use commercially available reference color panels to determine a color gamut of a biological sample for colorimetric diagnostics of interest. These bioassay reference color panels, such as dental shade guides, urine test strips, pH or peroxide strips, provide a narrow range of colors that can be used for gamut design. The color gamut can be determined by acquiring CIE xy chromaticity values of reference color panels using a white-light D65 LED light source. By converting RGB values to ground truth CIE XYZ and CIE xy chromaticity values, a unique and narrow triangular gamut can be defined based on the local cluster of reference color panels.

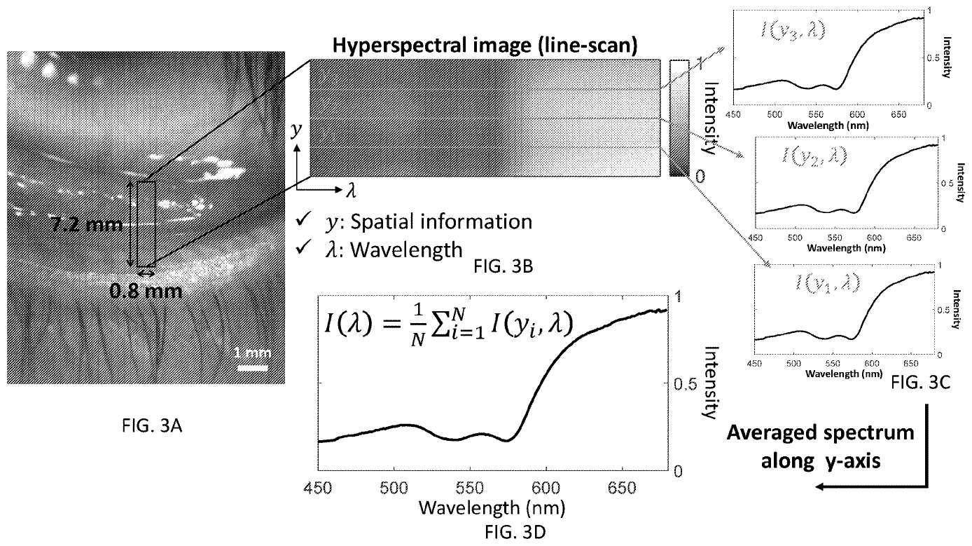

Each of these gamut processing approaches are now defined in more details. When there are no commercially available reference color panels for a biological target of interest, parametric biological tissue spectral modeling can be used to augment homogeneous data for proper gamut determination. For example, we focus on blood Hgb and peripheral tissue perfusion where blood Hgb is a dominant pigment. First, in this approach a plurality of hyperspectral images from a plurality of subjects from an area of interest is received. In the experiment conducted associated with the present disclosure 153 subjects were examined generating 153 hyperspectral images. An example of such a hyperspectral image is shown in FIGS. 3A-3C. As shown in FIG. 3A, a hyperspectral image capture device captures a line-scan from an area of interest (e.g., the inner eyelid). The resulting hyperspectral image is shown in FIG. 3B. This image is subdivided into three parts along a vertical (y-axis) while the horizontal axis (x-axis) is the associated wavelength in nm. Each of these subdivisions is shown as a spectrum in FIG. 3C relating intensity to wavelength. The spectra in FIG. 3C are averaged into a spectrum which is shown in FIG. 3D. Thus, for each of the 153 hyperspectral images, a spectrum is generated as shown in FIG. 3D. Next, a mathematical relationship is established between intensity as a function of wavelength, wherein the mathematical relationship includes a plurality of parameters. The mathematical relationship is expressed as:

$$I ( λ ) = [ b + ( 1 - b ) ( {{a}_{11} [ \frac{λ}{{λ}_{0}} ]}^{{a}_{12}} + {{a}_{13} [ \frac{λ}{{λ}_{0}} ]}^{{a}_{14}} ) ] {e}^{\{ - {a}_{2} {μ}_{a}^{Total} ( λ ) \times [ b + ( 1 - b ) \frac{{a}_{3} ( 1 - exp [ - {a}_{4} {μ}_{a}^{Total} ( λ ) ] )}{{a}_{4} {μ}_{a}^{Total} ( λ )} ] \}} , \qquad ( 1 )$$

wherein I represents spectral intensity,
λ represents wavelength,
the plurality of parameters are a₁₁, a₁₂, a₁₃, a₁₄, a₂, a₃, a₄, a₅, and a₆, wherein:
a₁₁, a₁₂, a₁₃, and a₁₄ represent tissue scattering contributions,
a₂ represents optical pathlength,
a₃ represents blood volume fraction,
a₄ represents effective blood vessel diameter in peripheral tissue,
a₅ represents blood hemoglobin (Hgb) content,
a₆ represents blood oxygen saturation,
λ₀ represents a constant for wavelength normalization,

$${μ}_{a}^{Total} ( λ )$$

represents total absorption coefficient including oxygenated and deoxygenated Hgb, wherein:

$${μ}_{a}^{Total} ( λ ) = {a}_{5} [ {a}_{6} {ε}_{{HgbO}_{2}} ( λ ) + ( 1 - {a}_{6} ) {ε}_{Hgb} ( λ ) ] , \qquad ( 2 )$$

wherein b represents a flag wherein when b=0, the mathematical relationship represents peripheral tissue and when b=1, the mathematical relationship represents whole blood,
ε_{HgbO}_{₂}(λ) represents extinction coefficient of oxygenated Hgb, and
ε_{Hgb}(λ) represents extinction coefficient of deoxygenated Hgb.

A set of possible spectral profiles of peripheral tissue (b=0) and whole blood (b=1) are synthesized using the generalized spectral equations of biological tissue, presented above (Equations 1 and 2).

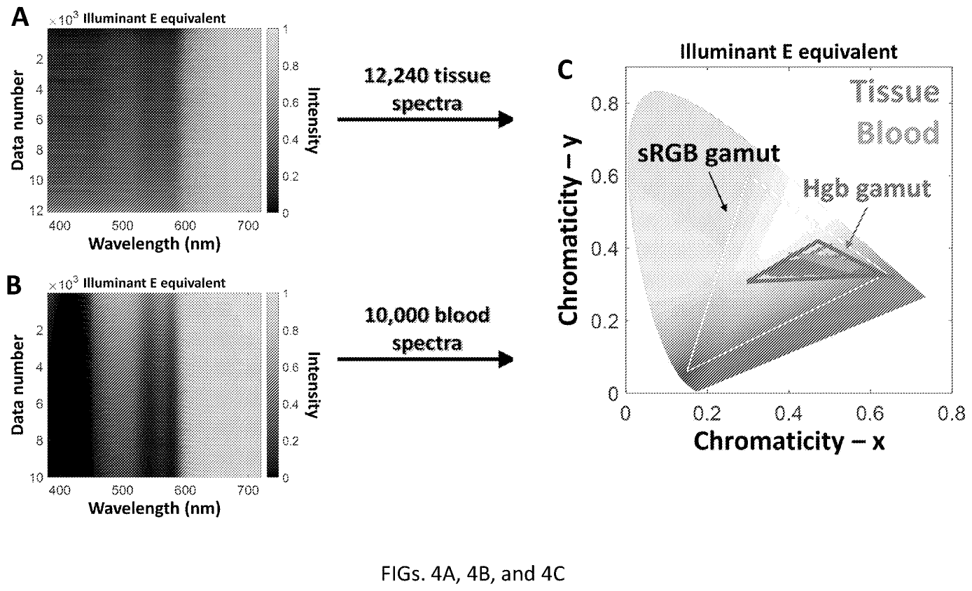

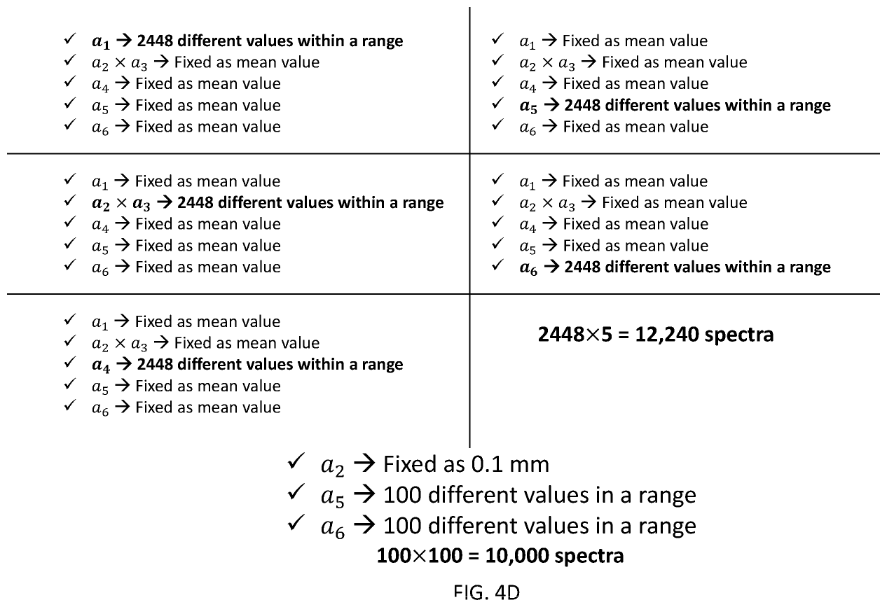

Table 1 summarizes the physiologically relevant ranges of the model parameters a₁₁, a₁₂, a₁₃, a₁₄, a₂, a₃, a₄, a₅, and a₆, reflecting the optical properties of peripheral perfusion and blood Hgb. By varying these parameters at specific intervals (Equations 1 and 2), we generate 12,240 spectral data of peripheral tissue shown in FIG. 4A and 10,000 spectral data of whole blood shown in FIG. 4B in the visible range. As a result, CIE XYZ and CIE xy chromaticity values are calculated from a total of 22,240 synthesized blood-related spectra, as related to this non-limiting example, using Equations 3 and 4. The division between the 12,240 and the 10,000 spectral data based on the plurality of parameters are shown in FIG. 4D.

TABLE 1
Physiologically relevant ranges of the model parameters in
spectral modeling of peripheral perfusion and blood Hgb samples

| | | Relevant range | | |
|---|---|---|---|---|
| | | Peripheral | | Whole |
| | | tissue | | blood |
| Parameter | Parameter description | (b = 0) | | (b = 1) |
| a₁₁ | Scattering amplitude | 0-1 | | N/A |
| a₁₂ | Scattering slope | −1-2 | | N/A |
| a₁₃ | Scattering amplitude | 0-1 | | N/A |
| a₁₄ | Scattering slope | −1-2 | | N/A |
| a₂ | Optical pathlength (mm) | a₂ × a₃ | 0.02-0.1 | 0.1 |

| | | | |
|---|---|---|---|
| a₃ | Blood volume fraction | | N/A |
| a₄ | Effective blood | 5-70 | N/A |
| | vessel diameter (μm) | | |
| a₅ | Blood Hgb level (g dL⁻¹) | 4-20 | |
| a₆ | Blood oxygen saturation | 0-1 | |

Using the specific set of spectral profiles, we determine the corresponding color gamut, we employ the CIE XYZ color space to analyze the physiologically relevant colorimetric range. Each synthesized spectrum S(λ) can be mapped to the corresponding CIE XYZ tristimulus values using the CIE XYZ color matching functions.

$$X = \frac{1}{K} {\sum }_{i = 1}^{N} L ( {λ}_{i} ) S ( {λ}_{i} ) \overline{x} ( {λ}_{i} ) , \qquad ( 3 )$$

$$Y = \frac{1}{K} {\sum }_{i = 1}^{N} L ( {λ}_{i} ) S ( {λ}_{i} ) \overline{y} ( {λ}_{i} ) ,$$

$$Z = \frac{1}{K} {\sum }_{i = 1}^{N} L ( {λ}_{i} ) S ( {λ}_{i} ) \overline{z} ( {λ}_{i} ) ,$$

wherein
λᵢ is the i^{th} wavelength discretized from 380 nm to 720 nm with a spectral interval of 1 nm (N=341),
x̅(λ), y̅(λ), and z̅(λ) are the CIE XYZ color matching functions,
L(λ) is the illuminant spectrum, and

$$K = {\sum }_{i = 1}^{N} L ( {λ}_{i} ) \overline{y} ( {λ}_{i} ) .$$

In particular, L(λ)=1 as S(λ) was computed under the assumption of CIE standard illuminant E (equal energy radiator).

Thus, each of the plurality of synthesized spectra is converted to a CIE XYZ tristimulus value thereby generating a digital tissue-specific gamut, as shown in FIG. 4C in the triangular area identified as the Hgb gamut within the CIE xy chromaticity space. Thus, the CIE xy chromaticity diagram (FIG. 4C) exhibits the chromaticity values from a total of 22,240 blood-related spectral data; forming a local cluster such that a unique and narrow triangular gamut can be defined with three primary points of the CIE xy chromaticity (FIG. 4C): (x, y)=(0.30, 0.31), (0.47, 0.42), and (0.63, 0.33). This gamut is herein referred to as the digital Hgb gamut. To further factor out the brightness in the CIE XYZ values, the CIE xy chromaticity can be calculated:

$$x = \frac{X}{X + Y + Z} , and \qquad ( 4 )$$

$$y = \frac{Y}{X + Y + Z} .$$

Next the generation of a ground truth color chart is described. From a physical size perspective, the desired physical dimension should be the sizes of typical business cards or credit cards. We impose the number of reference colors to be greater than 100, although other numbers may be possible.

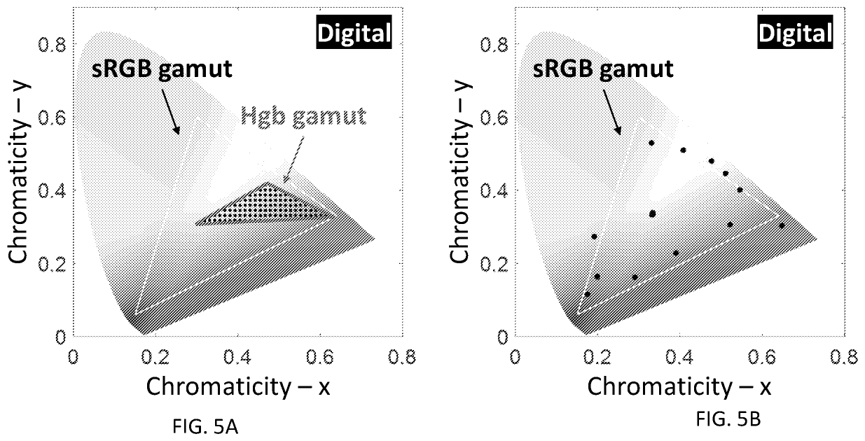

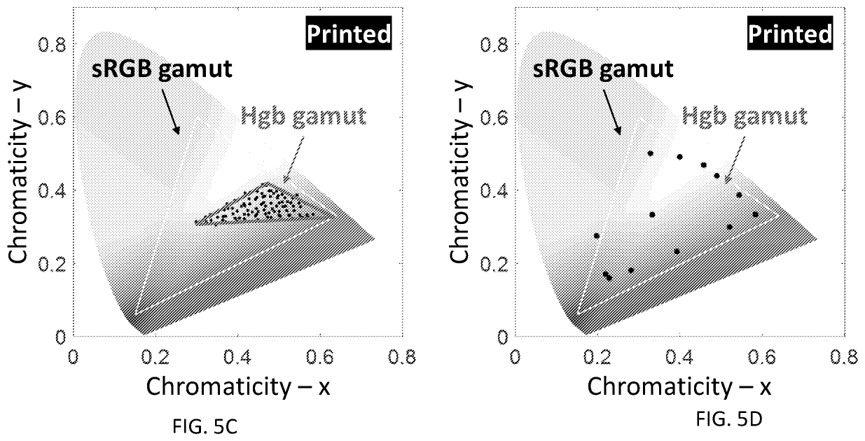

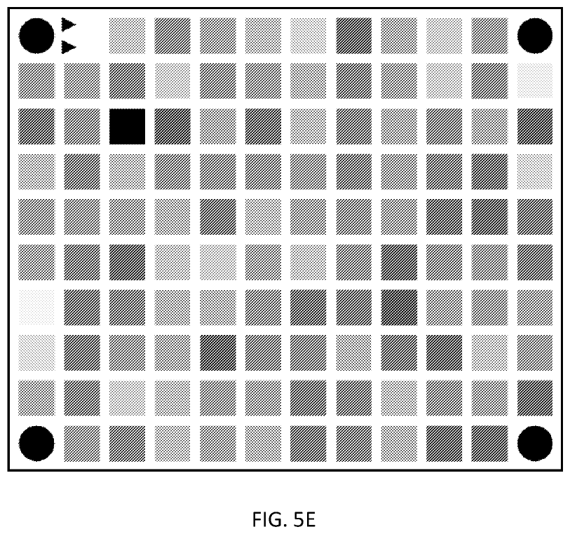

A number of points, e.g., 116, inside and outside of the digital Hgb gamut are chosen as shown in FIGS. 5A and 5B (these points represent 116 reference color patches including 100 CIE xy chromaticity values that are uniformly distributed inside the Hgb gamut as well as 12 primary colors and four black and white colors to interpolate colorimetric outliers outside the Hgb gamut and to compute the spectral response functions of the image capture device, e.g., a smartphone camera). Next, a printed tissue-specific gamut is determined based on the digital tissue-specific gamut, whereby each of the plurality of digital gamut points corresponds to an associated printed gamut point thereby establishing a printing calibration corresponding to a plurality of CMYK (cyan, magenta, yellow, and key (black)) values used for printing the associated printed gamut points and by minimizing the error signal based on a regression technique known to a person having ordinary skill in the art. The resulting printed gamut is shown in FIGS. 5C and 5D for the associated printed gamut points. Each of these printed gamut points then corresponds to a reference color patch in a color chart (shown in FIG. 5E), thus the color chart which is the ground truth color chart for the biological area of interest also has 116 patches.

For the gamut mapping process, we utilized a total of 729 colors derived from various combinations of CMYK color values. CMYK refers to the four fundamental ink pigments for color printing. These colors were generated based on the U.S. Web Coated (SWOP) v2, which served as the standard CMYK profile in the working space. Black (K), which mainly affects the luminance of colors, was set to 0, while the primary colors of cyan (C), magenta (M), and yellow (Y) were manipulated to consider a wide range of colors. Specifically, there were a comprehensive set of 729 possible digital inputs by combining the values of C, M, and Y, which encompass a range of [0%, 12.5%, 25%, 37.5%, 50%, 62.5%, 75%, 87.5%, 100%]. The 729 CMYK input values were printed using a professional photographic inkjet printer (imagePROGRAF PRO-1000, Canon). We also applied the International Color Consortium (ICC) profile and used the manufacturer-recommended genuine paper (Photo Paper Premium Fine Art Smooth, Canon). We measured the reflectance spectra of 729 printed outputs using a scientific laboratory spectrometer (VS140 VIS-NIR, Horiba Jobin Yvon Inc.). We calculated the CIE xy chromaticity values from the measured spectral data of printed colors. Consequently, the database of CMYK (digital input) and CIE xy chromaticity (printed output) for the 729 distinct colors were prepared for the gamut mapping.

The one-to-one mapping between the digital CMYK input and printed output values, enabling optimal color selection for desired printouts were based on two main steps:
1) We identified the u^{th} printed output values of the 729 colors such that the CIE xy chromaticity

$$( {XY}_{u}^{print} = {[ {x}_{u}^{print} , {y}_{u}^{print} ]}^{T} )$$

best matched that of the v^{th} color patch

$$( {XY}_{v}^{chart} = {[ {x}_{v}^{chart} , {y}_{v}^{chart} ]}^{T} )$$

by minimizing

$$‖ {XY}_{u}^{print} - {XY}_{v}^{chart} ‖ ,$$

where ∥·∥ denotes the Euclidean norm; and
2) Corresponding to the u^{th} printed output values, we selected the u^{th} digital CMYK input values, which were used for printing the desired physical reference color.

Physical charts were printed by a professional photographic inkjet printer, using the digital CMYK input values for the desired reference color patches. To further validate the printed color output values, we re-measured the reflectance spectra of all of the 116 color patches in the printed charts using a scientific laboratory spectrometer. CIE XYZ and CIE xy chromaticity values were calculated from the measured spectra using Equations 3 and 4. Consequently, we confirmed that the CIE xy chromaticity values were within the range of colors that can be produced by the Hgb gamut. In particular, the measured CIE XYZ values of the 116 color patches served as the ground truth.

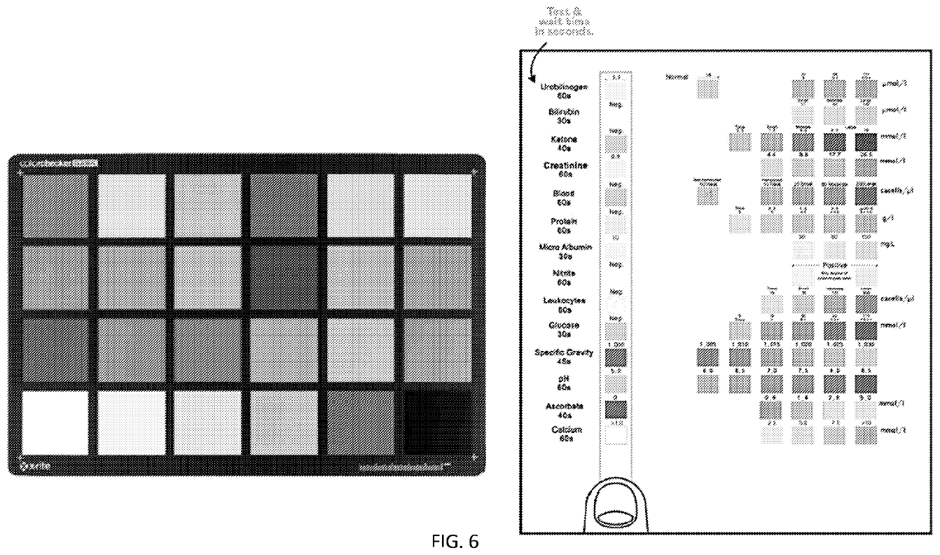

The second approach for receiving an RGB-based gamut is now described. When reference color panels are available for a specific diagnostic colorimetric application, these panels can be utilized for the determination of a relevant color gamut for a biological sample of interest. For example, dental shade guides consist of a comprehensive set of reference colors that mimic the natural shades of teeth, allowing for assessing colorimetric matches for tooth whitening procedure. Similarly, urine test strips incorporate a range of distinct colors that arise from different reagent tests, which enables health monitoring of vital organs such as kidney and liver, as well as measuring glucose levels. In addition, pH or peroxide strips exhibit unique colors corresponding to varying pH values and peroxide levels. Importantly, using these reference color panels, we can map out the homogeneous range of colorimetric characteristics of a target biological sample. Initially, a non-calibrated image of a bioassay-specific test strip is received along with a conventional color chart (e.g., Macbeth ColorChecker or X-Rite ColorChecker), wherein the color chart has m color patches, e.g., m=24, as shown by the example provided in FIG. 6. From the non-calibrated image, RGB information is acquired for the color chart and the test strip. Thus, an RGB matrix of 24×3 represents the color chart. This matrix is multiplied by a color-correction matrix of size 3×3, including 9 unknown parameters. The multiplication results in a calibrated matrix of 24×3 matrix which expresses a calibrated color information of the color chart in the CIE XYZ space. The calibrated color values of the color chart are known apriori. The 9 parameters of the 3×3 color-correction matrix are thus determined via known regression techniques, e.g., simple linear regression, by employing least square techniques, also known to a person having ordinary skill in the art. Once the 3×3 color-correction matrix is known, that matrix is multiplied by the matrix representing the test strip in order to determine a calibrated color information of the test strip in the CIE XYZ space.

Alternative approaches may be implemented for generating CIE XYZ tristimulus value based on converting an RGB-based matrix to a CIE XYZ matrix (m×3) and then multiplying by a second version of the color-correction matrix (3×3) to obtain a CIE XYZ matrix (m×3). Still in another alternative approach of generating a CIE XYZ tristimulus value is based on multiplying a first RGB-based matrix (m×3) by a third version of the color-correction matrix (3×3) to obtain a second RGB-based matrix (m×3) and then converting the second RGB-based matrix (m×3) to a CIE XYZ matrix (m×3).

Thus, the exact color gamut of a specific biological sample is determined by acquiring CIE xy chromaticity values of reference color panels using the most commonly used Macbeth ColorChecker (or X-Rite ColorChecker) consisting of 24 color patches. Specifically, single-shot RGB photos are captured for the reference color panels juxtaposed with Macbeth ColorChecker, under a CIE standard illuminant LED light source with color temperature of 6500 K (also known as D65). Given that CIE XYZ coordinates of 24 color patches in Macbeth ColorChecker under D65 LED illumination are apriori accessible, these coordinates can be used to derive an RGB-to-CIE XYZ conversion matrix, as described above. By employing the derived conversion matrix, RGB values are transformed into corresponding CIE XYZ values for the reference color panels under D65 LED illumination. CIE xy chromaticity values of the reference color panels are calculated from the CIE XYZ values. It should be noted that these chromaticity values form a local cluster in the CIE xy chromaticity diagram as discussed with reference to FIG. 4C. Consequently, we define a unique and narrow triangular gamut that is circumscribed about the cluster, having three primary points of CIE xy chromaticity, as discussed above with respect to FIG. 4C. The remainder of the steps are similar to spectroscopic gamut determination to generate the ground truth color chart as described with respect to FIGS. 5A-5E.

In both approaches, a ground truth color chart is provided containing a plurality of color patches along with ground truth color values of each of the color patches for various domains including RGB, CIE XYZ, CIE RGB, CIE LAB, CIE LUV, HSL, and HSV.

Among CIE standard illuminants (also known as reference illuminants), CIE illuminant E stands out as a theoretical reference with a 100% uniform spectral power distribution across the entire wavelength range (equal energy radiator or spectrally uniform illumination). Although CIE illuminant E is considered to exist solely in theory within the color science community, in the field of tissue optics and spectroscopy, spectral normalization involving a reflectance standard is frequently used to factor out the spectral response of the illumination and system, which is essentially equivalent to using CIE illuminant E. Given the availability of reflectance standards with a reflectivity of >99% over the entire visible range, spectral normalization is equivalent to using illuminant E.

We obtained the spectral intensity O(λ) reflected from a sample of interest under CIE illuminant E without using a physical light source of illuminant E as follows. The spectral intensity Iₘ(λ) reflected from the sample under an arbitrary light illumination, measured by a scientific laboratory spectrometer, can be expressed as a function of the wavelength of light A:

$${I}_{m} ( λ ) = L ( λ ) \cdot C ( λ ) \cdot D ( λ ) \cdot O ( λ ) , \qquad ( 5 )$$

where L(λ) is the spectral profile of the illumination light source,
C(λ) is the spectral response of all optical components in the system, and D(λ) is the spectral response function (also known as the spectral sensitivity) of the image sensor. Using a white diffuse (Lambertian) reflectance standard having a reflectivity of >99% in the visible range (SRT-99-050, Labsphere), the spectral intensity I_{ref}(λ) reflected from the white reflectance standard under the identical imaging setting as the sample can be obtained:

$${I}_{ref} ( λ ) = L ( λ ) \cdot C ( λ ) \cdot D ( λ ) . \qquad ( 6 )$$

Then, O(λ) is calculated by normalizing Iₘ(λ) by I_{ref}(λ):

$$O ( λ ) = \frac{{I}_{m} ( λ )}{{I}_{ref} ( λ )} . \qquad ( 7 )$$

Consequently, obtaining the spectral intensity reflected from an arbitrary sample under CIE illuminant E was straightforward, mimicking the scenario in which illuminant E was used. It should be noted that CIE illuminant E via spectral normalization allowed for the definition of the absolute colors of a sample because the spectral intensity reflected from the sample is not affected by the physical illumination source and acquisition conditions.

Those having ordinary skill in the art will recognize that numerous modifications can be made to the specific implementations described above. The implementations should not be limited to the particular limitations described. Other implementations may be possible.

1. A method of generating a ground truth color chart including color patches for biological imaging and color-based bioassays, comprising:
using a mathematical relationship associated with a tissue of interest expressing a relationship between reflection intensity as a function of wavelength, the received mathematical relationship having a plurality of parameters each with an established range;
synthesizing a plurality of synthesized spectra based on the mathematical relationship;
converting each of the plurality of synthesized spectra to a predetermined color space under a predetermined illuminant, thereby generating a digital tissue-specific gamut;
identifying a plurality of digital gamut points within the digital tissue-specific gamut based on a predetermined criterion; and
printing a tissue-specific gamut based on the digital tissue-specific gamut thus establishing a ground truth color chart having a plurality of color patches, whereby each of the plurality of digital gamut points corresponds to an associated printed color patch using a predetermined printing calibration.

2. The method of claim 1, wherein the predetermined illuminant is a standard illuminant established by the International Commission on Illumination (CIE).

3. (canceled)

4. The method of claim 2, wherein the tissue of interest includes at least one of skin, wound, teeth, tongue, eye, bulbar conjunctiva, palpebral conjunctiva, whole blood, eye tears, nailbed, external tissue surrounding eye, stool, urine, saliva, body fluid, and biological solutions.

5. (canceled)

6. (canceled)

7. (canceled)

8. (canceled)

9. (canceled)

10. (canceled)

11. (canceled)

12. (canceled)

13. (canceled)

14. The method of claim 2, wherein the step of synthesizing a plurality of synthesized spectra is based on a plurality of parameter data points associated with each of the parameters of the plurality of parameters.

15. The method of claim 2, wherein one or more of the plurality of parameters are associated with whole blood and one or more of the parameters are associated with peripheral tissue.

16. The method of claim 2, wherein the mathematical relationship is expressed as:
$$I ( λ ) = [ b + ( 1 - b ) ( {{a}_{11} [ \frac{λ}{{λ}_{0}} ]}^{{a}_{12}} + {{a}_{13} [ \frac{λ}{{λ}_{0}} ]}^{{a}_{14}} ) ] {e}^{\{ - {a}_{2} {μ}_{a}^{Total} ( λ ) \times [ b + ( 1 - b ) \frac{{a}_{3} ( 1 - exp [ - {a}_{4} {μ}_{a}^{Total} ( λ ) ] )}{{a}_{4} {μ}_{a}^{Total} ( λ )} ] \}} ,$$
wherein
I represents spectral intensity,
λ represents wavelength,
the plurality of parameters are a₁₁, a₁₂, a₁₃, a₁₄, a₂, a₃, a₄, a₅, and a₆, wherein:
a₁₁, a₁₂, a₁₃, and a₁₄ represent tissue scattering contributions,
a₂ represents optical pathlength,
a₃ represents blood volume fraction,
a₄ represents effective blood vessel diameter in peripheral tissue,
a₅ represents blood hemoglobin (Hgb) content,
a₆ represents blood oxygen saturation,
λ₀ represents a constant for wavelength normalization,
$${μ}_{a}^{Total} ( λ )$$
represents total absorption coefficient including oxygenated and deoxygenated Hgb, wherein:
$${μ}_{a}^{Total} ( λ ) = {a}_{5} [ {a}_{6} {ε}_{{HgbO}_{2}} ( λ ) + ( 1 - {a}_{6} ) {ε}_{Hgb} ( λ ) ] ,$$
wherein b represents a flag wherein when b=0, the mathematical relationship represents peripheral tissue and when b=1, the mathematical relationship represents whole blood,
ε_{HgbO}_{₂}(λ) represents extinction coefficient of oxygenated Hgb, and
ε_{Hgb}(λ) represents extinction coefficient of deoxygenated Hgb.

17. The method of claim 15, wherein the tissue of interest includes whole blood and peripheral tissue; a₁₁ is represented by a range of about 0 to about 1 for b=0; and not applicable for b=1; a₁₂ is represented by a range of about −1 to about 2 for b=0; and not applicable for b=1; a₁₃ is represented by a range of about 0 to about 1 for b=0; and not applicable for b=1; a₁₄ is represented by a range of about −1 to about 2 for b=0; and not applicable for b=1; a₂×a₃ is represented by a range of about 0.02 to about 0.1 for b=0; a₂ is about 0.1, and a₃ is not applicable for b=1; a₄ is represented by a range of about 5 to about 70 for b=0; and not applicable for b=1; a₅ is represented by a range of about 0 to about 20; and a₆ is represented by a range of about 0 to about 1.

18. The method of claim 15, wherein a₁₁, a₁₂, a₁₃, a₁₄, a₂, a₃, a₄, a₅, and a₆ are related to peripheral tissue for when b=0.

19. The method of claim 15, wherein a₂, a₅, and a₆ are related to whole blood for when b=1, where
$${μ}_{a}^{Total} ( λ )$$
represents total absorption coefficient including other light absorbing pigments and molecules in biological samples.

20. The method of claim 2, wherein the predetermined color space is at least one of RGB, CIE XYZ, CIE RGB, CIE YUV, CIE UVW, CIE LAB, CIE LCH, CIE LUV, HSL, HSV, HCL, LMS, OKLAB, OKLCH, and YCbCr.

21. (canceled)

22. (canceled)

23. (canceled)

24. (canceled)

25. (canceled)

26. (canceled)

27. (canceled)

28. (canceled)

29. (canceled)

30. (canceled)

31. A method of generating a ground truth color chart for color-based bioassays, comprising:
generating a digital bioassay-specific gamut using a predetermined color space under a predetermined illuminant for a bioassay-specific test strip having a plurality of reference color panels;
identifying a plurality of digital gamut points within the digital bioassay-specific gamut based on a predetermined criterion; and
printing a bioassay-specific gamut based on the digital bioassay-specific gamut thus establishing a ground truth color chart having a plurality of color patches, whereby each of the plurality of digital gamut points corresponds to an associated printed color patch using a predetermined printing calibration.

32. The method of claim 30, wherein the predetermined illuminant is a standard illuminant established by the International Commission on Illumination (CIE).

33. The method of claim 32, wherein the bioassay-specific test strip is at least for one of urine, blood, eye tears, saliva, stool, body fluid, and biological solutions.

34. (canceled)

35. (canceled)

36. (canceled)

37. (canceled)

38. (canceled)

39. (canceled)

40. The method of claim 32, wherein the predetermined color space is at least one of RGB, CIE XYZ, CIE RGB, CIE YUV, CIE UVW, CIE LAB, CIE LCH, CIE LUV, HSL, HSV, HCL, LMS, OKLAB, OKLCH, and YCbCr.

41. (canceled)

42. (canceled)

43. (canceled)

44. (canceled)

45. (canceled)

46. (canceled)

47. (canceled)

48. (canceled)

49. (canceled)

50. (canceled)
